(19) United States
(12) Patent Application Publication
(10) Pub. No.: US 2020/0260944 A1
(43) Pub. Date: Aug. 20, 2020

(54) METHOD AND DEVICE FOR RECOGNIZING MACULAR REGION, AND COMPUTER-READABLE STORAGE MEDIUM

(71) Applicant: Baidu Online Network Technology (Beijing) Co., Ltd., Beijing (CN)

(72) Inventors: Qinpei SUN, Beijing (CN); Yehui YANG, Beijing (CN); Lei WANG, Beijing (CN); Yanwu XU, Beijing (CN); Yan HUANG, Beijing (CN)

(73) Assignee: Baidu Online Network Technology (Beijing) Co., Ltd., Beijing (CN)

(21) Appl. No.: 16/698,673

(22) Filed: Nov. 27, 2019

(30) Foreign Application Priority Data
Feb. 19, 2019 (CN) 201910164878.1

(51) Int. Cl. A61B 3/00; A61B 3/12; G06T 7/00; G06N 7/00; G06N 20/00

(52) U.S. Cl. CPC A61B 3/0025; A61B 3/12; G06T 7/0012; G06T 2207/20081; G06N 20/00; G06T 2207/30041; G06T 2207/30101; G06N 7/005

(57) ABSTRACT

A method and a device for recognizing a macular region and a computer-readable storage medium are provided. The method includes: obtaining a fundus image of a target object; extracting blood vessel information and optic disc information from the fundus image; inputting the blood vessel information and the optic disc information into a regression model to obtain location information of a macular fovea; and determining location information of the macular region of an eye of the target object, based on the location information of the macula fovea. In the embodiments of the application, the problem that the macular region cannot be accurately recognized when the image quality of the macular region is impaired is solved.

### CROSS-REFERENCE TO RELATED APPLICATIONS

This application claims priority to Chinese Patent Application No. 201910164878.1, and filed on Mar. 5, 2019, which is hereby incorporated by reference in its entirety.

### TECHNICAL FIELD

The present disclosure relates to the field of medical image processing technology, and in particular, to a method and a device for recognizing a macular region.

### BACKGROUND

A macular region is located in a center of a retina and is the most sensitive region of vision. Cone cells responsible for vision and color vision are distributed in the region. Therefore, any lesion involving the macula will cause a significant decrease in central vision, darkness and deformation of an object. The macular region has no clear boundary. And a region extracted based on the macula fovea is referred to as the macular region.

At present, the extraction of the macular region is generally solved by the following two schemes. In the first scheme, an image processing threshold method is used to locate the macula fovea by using the low brightness of the macula fovea, and then the macular region is extracted. In the second scheme, morphological and feature extraction techniques are used to locate a center of the macula, and the macular region is finally extracted.

However, in the above-mentioned schemes, there is an issue when the image quality of the macular region is impaired, so that the macular region cannot be accurately recognized.

### SUMMARY

A method and a device for recognizing a macular region are provided according to embodiments of the present disclosure, so as to at least solve one or more technical problems in the existing technology.

In a first aspect, a method for recognizing a macular region is provided according to an embodiment of the present application, the method includes:

obtaining a fundus image of a target object;

extracting blood vessel information and optic disc information from the fundus image;

inputting the blood vessel information and the optic disc information into a regression model to obtain location information of a macular fovea; and

determining region location information of the macular region of an eye of the target object, based on the location information of the macula fovea.

In one possible implementation, the method further includes:

obtaining at least one historical fundus image;

obtaining historical blood vessel information, historical optic disc information, and historical location information of the macular fovea based on the at least one historical fundus image; and

training the regression model by taking the historical blood vessel information and the historical optic disc information as input parameters of the regression model and taking the historical location information of the macular fovea as an output parameter of the regression model.

In one possible implementation, the historical blood vessel information includes: historical coordinates of a blood vessel barycenter, and historical coordinates of a convergence point of at least two blood vessels, and

the historical optic disc information includes: a historical center of an optic disc, a historical horizontal diameter of the optic disc, and a historical vertical diameter of the optic disc.

In one possible implementation, the extracting blood vessel information and optic disc information from the fundus image includes:

determining a center of the optic disc, a horizontal diameter of the optic disc and a vertical diameter of the optic disc based on the fundus image;

obtaining a location information set of a blood vessel from the fundus image, wherein the location information in the location information sets of different blood vessels is at least partially different; and

determining coordinates of a blood vessel barycenter of the blood vessel based on the location information set of the blood vessel, and determining coordinates where an overlapping region of multiple blood vessels is the largest, as coordinates of a convergence point.

In one possible implementation, the determining location information of the macular region of an eye of the target object, based on the location information of the macula fovea includes:

determining a radius of the macular region based on the optic disc information; and

determining the location information of the macular region by taking the location information of the macula fovea as a center point based on the radius of the macular region.

In one possible implementation, the method further includes:

generating a mask based on the location information of the macular region of the eye of the target object; and

obtaining an image at the macular region of the eye of the target object based on the mask and the fundus image of the target object.

In a second aspect, a device for recognizing a macular region is provided according to an embodiment of the present application, the device includes:

an information obtaining unit, configured to obtain a fundus image of a target object;

and extract blood vessel information and optic disc information from the fundus image;

a model processing unit, configured to inputting the blood vessel information and the optic disc information into a regression model to obtain location information of a macular fovea;

and

an information recognizing unit, configured to determine location information of the macular region of an eye of the target object, based on the location information of the macula fovea.

In one possible implementation, the model processing unit is configured to:

obtain at least one historical fundus image; obtain historical blood vessel information, historical optic disc information, and historical location information of the macular fovea based on the at least one historical fundus image; and train the regression model by taking the historical blood vessel information and the historical optic disc information as input parameters of the regression model and taking the historical location information of the macular fovea as an output parameter of the regression model.

In one possible implementation, the historical blood vessel information includes: historical coordinates of a blood vessel barycenter, and historical coordinates of a convergence point of at least two blood vessels, and

the historical optic disc information includes: a historical center of an optic disc, a historical horizontal diameter of the optic disc, and a historical vertical diameter of the optic disc.

In one possible implementation, the information obtaining unit is configured to:

determine a center of the optic disc, a horizontal diameter of the optic disc and a vertical diameter of the optic disc based on the fundus image; obtain a location information set of a blood vessel from the fundus image, wherein the location information in location information sets of the different blood vessels is at least partially different; and determine coordinates of a blood vessel barycenter of the blood vessel based on the location information set of the blood vessel, and determining coordinates where an overlapping region of multiple blood vessels is the largest, as coordinates of a convergence point.

In one possible implementation, the information recognizing unit is configured to:

determine a radius of the macular region based on the optic disc information; and

determine the location information of the macular region by taking the location information of the macula fovea as a center point based on the radius of the macular region.

In one possible implementation, the device further includes:

an image extracting unit, configured to generate a mask based on the location information of the macular region of the eye of the target object; and obtain an image at the macular region of the eye of the target object based on the mask and the fundus image of the target object.

In a third aspect, a device for recognizing a macular region is provided according to an embodiment of the present application, the device includes:

one or more processors; and

a storage device configured for storing one or more programs, wherein

the one or more programs are executed by the one or more processors to enable the one or more processors to implement any one of the above methods.

In a possible design, a device for recognizing a macular region includes a processor and a storage, the storage is configured to store a program for supporting the above method executed by the above device, the processor is configured to execute the program stored in the storage. The device further includes a communication interface configured for communication between the device and another apparatus or communication network.

In a fourth aspect, a computer-readable storage medium is provided according to an embodiment of the present application, for storing computer software instructions used by the device for recognizing a macular region, the computer software instructions include programs involved in execution of the above method for recognizing a macular region.

One of the above technical solutions has the following advantages or beneficial effects:

blood vessel information and optic disc information are extracted from the fundus image of the target object, the blood vessel information and optic disc information are input into the regression model, and location information of the macular region of an eye of the target object is determined based on the location information of the macula fovea. In this way, the location of the macular fovea can be obtained by using the regress algorithm according to the blood vessel information and optic disc information, which can effectively avoid the problem of the inability to recognize the macular region caused by the influence of illumination and lesion damage on the image quality of the macular region. The accuracy is improved, and the robustness of the extraction algorithm of the macular region is enhanced by using the above solutions.

The above summary is for the purpose of the specification only and is not intended to be limiting in any way. In addition to the illustrative aspects, embodiments, and features described above, further aspects, embodiments, and features of the present application will be readily understood by reference to the drawings and the following detailed description.

### BRIEF DESCRIPTION OF THE DRAWINGS

In the drawings, unless otherwise specified, identical reference numerals will be used throughout the drawings to refer to identical or similar parts or elements. The drawings are not necessarily drawn to scale. It should be understood that these drawings depict only some embodiments disclosed in accordance with the present application and are not to be considered as limiting the scope of the present application.

FIG. 1 shows a schematic flowchart of a first method for recognizing a macular region according to an embodiment of the present application;

FIG. 2 shows a schematic flowchart of a second method for recognizing a macular region according to an embodiment of the present application;

FIG. 3 shows a schematic flowchart of a third method for recognizing a macular region according to an embodiment of the present application;

FIG. 4 shows a schematic diagram of blood vessel information in a fundus image;

FIG. 5 shows a schematic diagram of macular region and optic disc location in a fundus image;

FIG. 6 shows a schematic flowchart of a method for extracting an image of a macular region according to an embodiment of the present application;

FIG. 7 shows a schematic diagram of extracting an image of a macular region from a fundus image based on a mask according to an embodiment of the present application;

FIG. 8 shows a schematic flowchart of a method for recognizing a macular region and extracting a macular region according to an embodiment of the present application;

FIG. 9 shows a schematic diagram of images in various stages of a process according to an embodiment of the present application;

FIG. 10 shows a structural block diagram of a first device for recognizing macular region according to an embodiment of the present application;

FIG. 11 shows a structural block diagram of a second device for recognizing macular region according to an embodiment of the present application.

### DETAILED DESCRIPTION

In the following, only certain exemplary embodiments are briefly described. As those skilled in the art would realize, the described embodiments may be modified in various different ways, all without departing from the spirit or scope of the present application. Accordingly, the drawings and description are to be regarded as illustrative in nature and not restrictive.

In an embodiment, FIG. 1 shows a flowchart of a method for recognizing a macular region according to an embodiment of the present application, the method includes:

S11: obtaining a fundus image of a target object;

S12: extracting blood vessel information and optic disc information from the fundus image;

S13: inputting the blood vessel information and the optic disc information into a regression model to obtain location information of a macular fovea; and

S14: determining location information of the macular region of an eye of the target object, based on the location information of the macula fovea.

The solution provided in this embodiment can be applied to an apparatus having an image analysis and processing function, such as a terminal apparatus, and can also be applied to a network apparatus.

When the solution is applied to the terminal apparatus, the fundus image of the target object can be acquired by an image acquisition unit provided on the terminal apparatus. Then, a processing unit of the terminal apparatus performs the foregoing S11 to S14 to obtain the macular region of the eye of the target object.

When the solution is applied to a network apparatus, the fundus image of the target object acquired and sent by the terminal apparatus with the acquisition unit may be received, and then the network apparatus performs S11 to S14. When the solution is applied on the network side, a recognition result of the macular region of the eye of the target object may be transmitted to the terminal apparatus by the network apparatus after performing S14. This embodiment does not limit how to acquire and how to obtain the fundus image of the target object. Therefore, the above various steps are specifically described only for the terminal apparatus or the network apparatus.

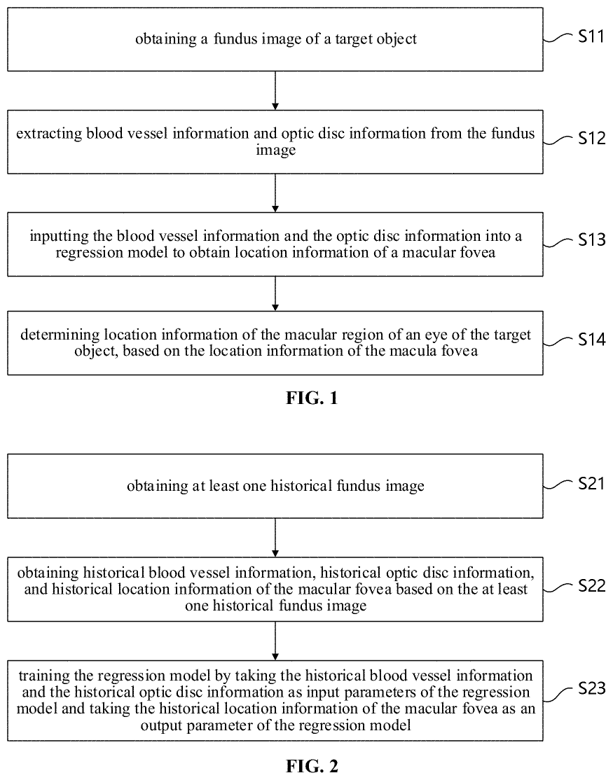

The solution provided by this embodiment will train the regression model before performing S11. With reference to FIG. 2, the process includes:

S21: obtaining at least one historical fundus image;

S22: obtaining historical blood vessel information, historical optic disc information, and historical location information of the macular fovea based on the at least one historical fundus image;

S23: training the regression model by taking the historical blood vessel information and the historical optic disc information as input parameters of the regression model and taking the historical location information of the macular fovea as an output parameter of the regression model.

The at least one historical fundus image may be from the same user or from different users; or may be N historical fundus images from one user and M historical fundus images from another user. The way to obtain at least one historical fundus image may be to obtain a plurality of historical fundus images that have been acquired and stored in a database.

In order to locate the macula fovea and extract the Region of Interest (ROI) of the macular region, additional information, such as blood vessel information and optic disc information, are needed.

The optic disc is a portion of a retina on which visual fibers converge and pass through an eyeball, and is generally a pale red elliptical structure with a clear boundary, in the fundus image. And a retinal blood vessel at the bottom of the eyeball is the only one part that can be observed non-invasively and directly, in the whole blood vessel system. Changes of the retinal blood vessel at the bottom of the eyeball, such as the blood vessel width, angle, and branch morphology and the like, can be used as a basis for diagnosing diseases related to the blood vessel. Ophthalmological blindness diseases, such as glaucoma, diabetic retinopathy, and age-related macular degeneration, can be directly observed from retinal vasculopathy. The location of the macular fovea is closely related to the location of the optic disc and distribution of blood vessels.

Therefore, in the solution provided in the embodiment, the historical optic disc information and the historical blood vessel information closely related to the location of the macular fovea in the historical fundus image are used to train the regression model.

Specifically, the historical optic disc information and the historical blood vessel information are extracted, which are closely related to the location of the macular fovea, with reference to S22, that is, the obtaining historical blood vessel information, historical optic disc information, and historical location information of the macular fovea based on the at least one historical fundus image.

The historical blood vessel information includes: historical coordinates of a blood vessel barycenter, and historical coordinates of a convergence point of at least two blood vessels; and the historical optic disc information includes: a historical center of an optic disc, a historical horizontal diameter of the optic disc, and a historical vertical diameter of the optic disc.

For example, the historical optic disc information of the historical fundus image may include a historical horizontal diameter of the optic disc dₕ, a historical vertical diameter of the optic disc dᵥ, a historical diameter of the optic disc ODD, and a historical center of the optic disc (x_{disc}, y_{disc});

The historical blood vessel information of the historical fundus image includes: historical coordinates of a blood vessel barycenter, which can be expressed as (xᵥₑₛₛₑₗ, yᵥₑₛₛₑₗ), historical coordinates of a convergence point of at least two blood vessels, such as coordinates of a convergence point of four aortas and a main vein in the blood vessels, which can be expressed as (x_{convergence}, y_{convergence}).

For obtaining historical optic disc information of a historical fundus image, the convolutional neural network (CNN) can be used to detect the optic disc to obtain the bounding-box of the optic disc, thereby obtaining a horizontal diameter of the optic disc, a vertical diameter of the optic disc, and coordinates of a center of the optic disc.

The method for obtaining historical blood vessel information may include: using a CNN semantic segmentation algorithm to perform pixel-level segmentation on blood vessels in the historical fundus image, that is, extracting at least one pixel including a blood vessel from the fundus image; and then obtaining a coordinate set of a blood vessel, which may be called a Mask of the blood vessel, based on coordinates of at least one pixel containing the blood vessel; extracting historical blood vessel information through the Mask of the blood vessel.

It should be noted that the mask of the blood vessel may be a respective coordinate set of each blood vessel in the historical fundus image, or may be coordinate sets of all blood vessels in the historical fundus image.

Further, the extracting the historical blood vessel information based on the mask of the blood vessel is: calculating coordinates of a sub-barycenter of each blood vessel according to a respective coordinate set of each blood vessel, and taking an average of the coordinates of sub-barycenters of all the blood vessels as historical coordinates of a blood vessel barycenter, wherein the calculation of coordinates of a sub-barycenter of each blood vessel may be performed by taking an average of pixel locations, and the obtained result is coordinates of a sub-barycenter.

Alternatively, historical coordinates of a blood vessel barycenter may be obtained by calculating an average of all the pixel locations using a coordinate set of pixel locations including all the blood vessels.

The method for obtaining historical coordinates of a convergence point may include selecting a location with the largest overlapping region or the most pixels as historical coordinates of a convergence point, according to the coordinates of the at least one blood vessel, such as the coordinates of the arterial and venous blood vessels.

The method for determining the historical location of the macular fovea can be manually labeled, and so on, and is not exhaustively illustrated here.

Further, the regression model can be trained by using the historical center of the optic disc in the historical fundus image, the historical horizontal diameter of the optic disc and the historical vertical diameter of the optic disc, the historical blood vessel information, and the historical information of the macular fovea, and expressed by the following formula:

Coordinate_{fovea}=f(Coordinate_{disc},Coordinateᵥₑₛₛₑₗ),

Where, f ( ) is an expression for the regression model, Coordinate_{disc} is information related to the optic disc, Coordinateᵥₑₛₛₑₗ is blood vessel coordinate information, and Coordinate_{fovea} is the coordinate of the macular fovea.

For example, in a case that the regression model is a selective polynomial regression, and the expression of the macular fovea regression model in the center of the optic disc is:

x_{fovea}=a₀+a₁x_{disc}+a₂dₕ+a₃dᵥ+a₄xᵥₑₛₛₑₗ+a₅x_{Convergence} (1)

y_{fovea}=b₀+b₁y_{disc}+b₂dₕ+b₃dᵥ+b₄yᵥₑₛₛₑₗ+b₅y_{Convergence} (2)

where, the variable (x_{disc}, y_{disc}) is the historical center of the optic disc, dₕ is the historical horizontal diameter of the optic disc, dᵥ is the historical vertical diameter of the optic disc, (xᵥₑₛₛₑₗ, yᵥₑₛₛₑₗ) is the historical coordinates of the blood vessel barycenter, and (x_{Convergence}, y_{Convergence}) is the historical coordinates of the convergence point of the blood vessels. (x_{fovea}, y_{fovea}) is the historical coordinates of the macular fovea.

Formula 1 and formula 2 can be trained based on the above information to obtain a₀˜a₅ and b₀˜b₅. The finally obtained a₀˜a₅ and b₀˜b₅ can be used as parameters in the trained regression model.

It should be understood that in the above training process, each time, one historical fundus image may be used to train the above formulas; then another historical fundus image is used to train the above formulas, until finally the a₀˜a₅ and b₀˜b₅ are obtained. Further, the method for determining whether a₀˜a₅ and b₀˜b₅ in the formulas 1 and 2 are successfully trained, may include: determining the training for the regression model is completed, when a₀˜a₅ and b₀˜b₅ are no longer changed by using the historical fundus image for consecutive N times. Certainly, there may be other ways to determine whether the regression model is successfully trained, but it is not exhaustively illustrated in this embodiment.

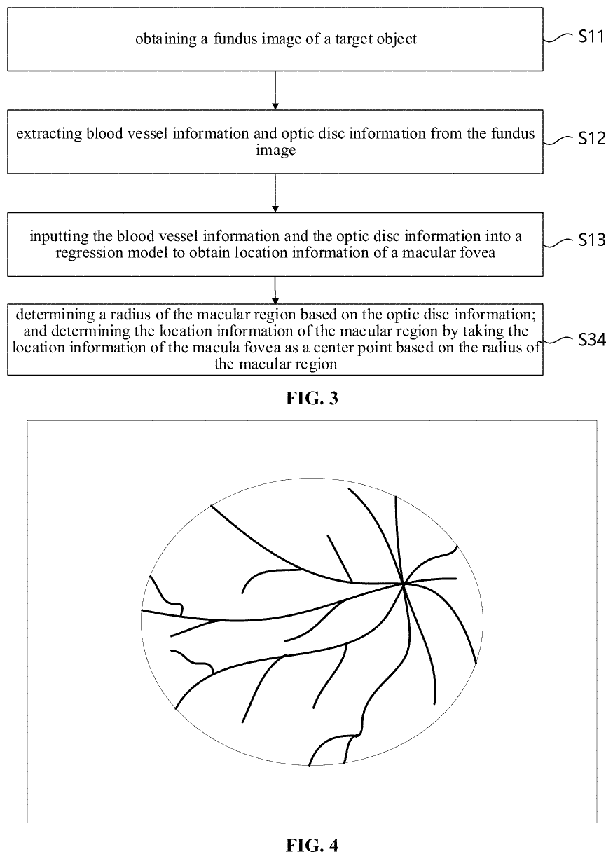

In another embodiment, FIG. 3 shows a schematic flowchart of a method for recognizing a macular region according to an embodiment of the present application, the method includes:

S11: obtaining a fundus image of a target object;

S12: extracting blood vessel information and optic disc information from the fundus image;

S13: inputting the blood vessel information and the optic disc information into a regression model to obtain location information of a macular fovea;

S34: determining a radius of the macular region based on the optic disc information; and determining the location information of the macular region by taking the location information of the macula fovea as a center point based on the radius of the macular region.

The regression model in this embodiment is a regression model trained in the foregoing embodiment, and the specific training method is omitted herein.

In the above S12, the extracting blood vessel information and optic disc information from the fundus image includes:

determining a center of the optic disc, a horizontal diameter of the optic disc and a vertical diameter of the optic disc based on the fundus image;

obtaining a location information set of a blood vessel from the fundus image, wherein the location information in location information sets of different blood vessels is at least partially different; and

determining coordinates of a blood vessel barycenter of the blood vessel based on the location information set of the blood vessel, and determining coordinates where an overlapping region of multiple blood vessels is the largest, as coordinates of a convergence point.

The center of the optic disc, the horizontal diameter of the optic disc, and the vertical diameter of the optic disc may constitute the optic disc information. In addition, the coordinates of the blood vessel barycenter of the blood vessel and the coordinates of the convergence point may be taken as the blood vessel information.

The method for obtaining the center of the optic disc, the horizontal diameter of the optic disc, and the vertical diameter of the optic disc is the same as the method for obtaining the historical center of the optic disc, the historical horizontal diameter of the optic disc, and the historical vertical diameter of the optic disc obtained in the foregoing embodiment. For example, through the CNN detection algorithm, the coordinates of the center of the optic disc, and the horizontal diameter and the vertical diameter of the optic disc can be obtained.

A plurality of blood vessels in the fundus image are shown in FIG. 4. The method for obtaining the blood vessel information may be the same as the method for obtaining the historical blood vessel information, for example, using a CNN semantic segmentation algorithm to segment a blood vessel in a fundus image into pixels to obtain the location of the pixel. The blood vessel information is determined based on the location.

The difference from the above-described training a regression model is that, in the embodiment, the blood vessel information and the optic disc information are directly input into the trained regression model to obtain the coordinates of the macular fovea. The model obtained by the training is used to regress the coordinates (x_{fovea}, y_{fovea}) of the macula fovea.

In S34, the radius of the macular region is determined based on the information of the optic disc in the fundus image. The radius of the macular region may be determined based on the diameter of the optic disc in the information of the optic disc; for example, the diameter of the optic disc is directly used as the radius of the macular region.

It should be noted that the diameter of the optic disc may include the horizontal diameter of the optic disc and the vertical diameter of the optic disc, and therefore, it is necessary to firstly determine the diameter of the optic disc. The manner of determining the diameter of the optic disc in this embodiment may include one of the following:

taking the maximum value of the horizontal diameter of the optic disc and the vertical diameter of the optic disc as the optic disc diameter;

calculating an average value of the horizontal diameter of the optic disc and the vertical diameter of the optic disc, and taking the average value as the optic disc diameter;

taking any one of the horizontal diameter of the optic disc and the vertical diameter of the optic disc as the optic disc diameter.

In S34, the location information of the macular fovea is taken as a center point, and the location information of the macular region is determined based on the center point and the radius of the macular region, which can be expressed as the center point (x_{fovea}, y_{fovea}), and the radius of the macular region ODD. Based on the center point and the radius of the macular region, the circular region is calculated and obtained, that is, the region of interest (ROI) of the macular region. For example, with reference to FIG. 5, the right side in the figure shows the optic disc 51, and the macular region obtained after the processing based on the foregoing steps may be the circular region 52 in the figure.

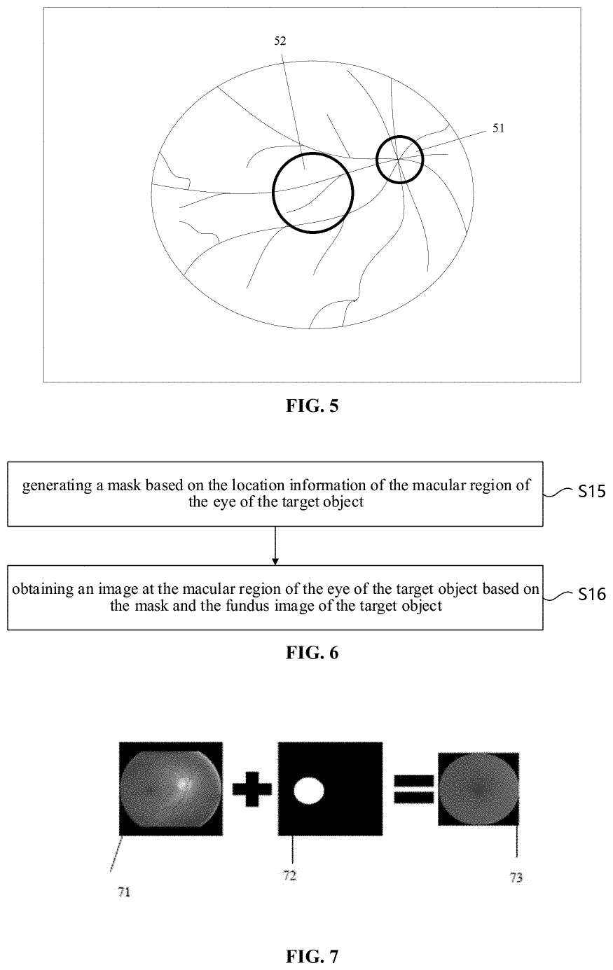

With reference to FIG. 6, a further process is provided in an embodiment, which includes:

S15: generating a mask based on the location information of the macular region of the eye of the target object;

S16: obtaining an image at the macular region of the eye of the target object based on the mask and the fundus image of the target object.

In other word, the mask of the ROI is generated for the location information of the macular region, that is, the circular region, and the region of interest of the macular region is extracted in combination with the fundus image.

The mask may be generated by generating a picture based on the location information of the macular region; the size (or dimension) of the mask may be the same as the fundus image; and in the mask, the region corresponding to the location information of the macular region in the fundus image is set to be a transparent display region, the remaining region is set to be a non-transparent display region.

The obtaining an image at the macular region of the eye of the target object based on the mask and the fundus image of the target object may be understood that after the mask is covered on the fundus image of the target object, the partial image of the fundus image displayed through the transparent display region is the macular region of the eye to the target object. For example, with reference to FIG. 7, the mask 72 is covered on the fundus image 71, and an image 73 containing only the macular region is obtained through the transparent display region of the mask.

The solution provided in this embodiment can also be applied to an apparatus having an image analysis and processing function, such as a terminal apparatus, and certainly, can also be applied to a network apparatus.

When the solution is applied to the terminal apparatus, the fundus image of the target object may be acquired by the image acquisition unit provided on the terminal apparatus. And then the processing unit of the terminal apparatus performs the foregoing S11 to S14 and S15 to S16 to obtain the image of the macular region of the eye of the target object.

When the solution is applied to the network apparatus, the fundus image of the target object acquired and sent by the terminal apparatus with the acquisition unit may be received, and then the network apparatus performs S11 to S14; further, when the solution is applied on the network side, after performing S14 and S15 to S16 to finally obtain the image of the macular region of the eye of the target object, the image of the macular region of the eye of the target object may be transmitted to the terminal apparatus by the network apparatus.

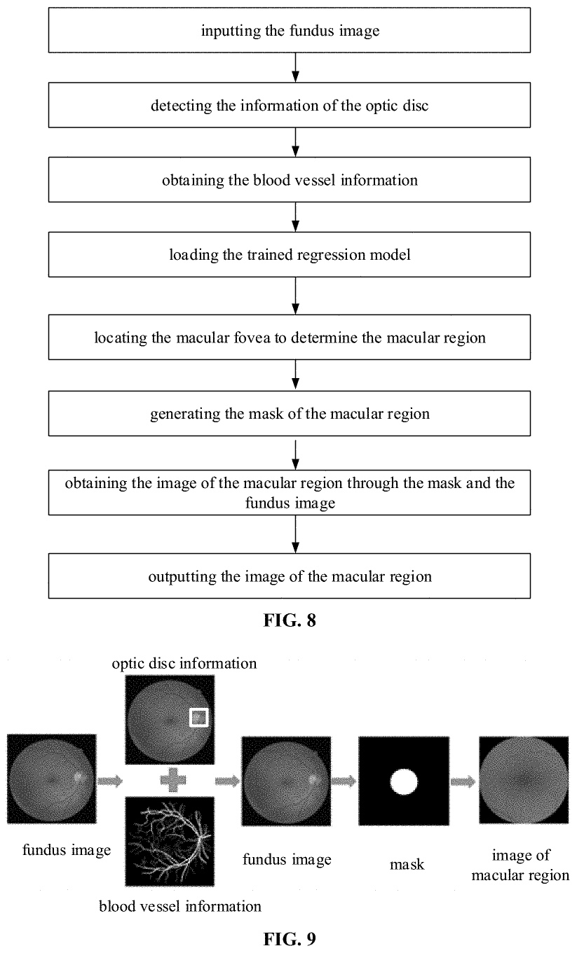

With reference to FIGS. 8 and 9, a specific embodiment is shown, which includes: detecting the information of the optic disc based on the fundus image after obtaining the input fundus image, wherein the information of the optic disc includes the horizontal and vertical diameters of the optic disc, and the center location of the optic disc; obtaining the blood vessel information based on the fundus image, wherein the blood vessel information includes the coordinates of the blood vessel barycenter and the coordinates of the convergence point; determining the location information of the macular fovea based on the output of the regression model, after inputting the information of the optic disc and the blood vessel information into the trained regression model; and then determining the location information of the macular region according to the location information of the macular fovea; generating the mask of the macular region according to the location information of the macular region; obtaining the image of the macular region through the mask and the fundus image; and outputting the image of the macular region finally.

The key to computer-aided diagnosis of macular degeneration is to extract the region of interest (ROI) from the fundus examination image. However, the location of the macular region is difficult, because the macular region itself has no clear boundary with other regions in the fundus. It is difficult to extract the ROI of the macular region by segmentation. The macular fovea is darker under the ophthalmoscope and there is a visible reflective point in the fovea. For the image with the distinct fovea, the ROI of the region can be extracted by using the location of the fovea. However, the fovea is susceptible to some lesions. For most of the images with the pathological macular region, it is difficult to distinguish the specific location of the fovea.

In the embodiment, by extracting the blood vessel information and the optic disc information in the fundus image of the target object, the blood vessel information and the optic disc information are input into the regression model to obtain the location information of the fovea of the macular region, and the location information of the macular region is determined based on the location information of the fovea of the macular region. In this way, the location of the macular fovea can be obtained by using the regress algorithm according to the blood vessel information and the optic disc information, which can effectively avoid the influence of illumination and lesion damage on the image quality of the macular region. The accuracy is improved, and the robustness of the extraction algorithm of the macular region is enhanced.

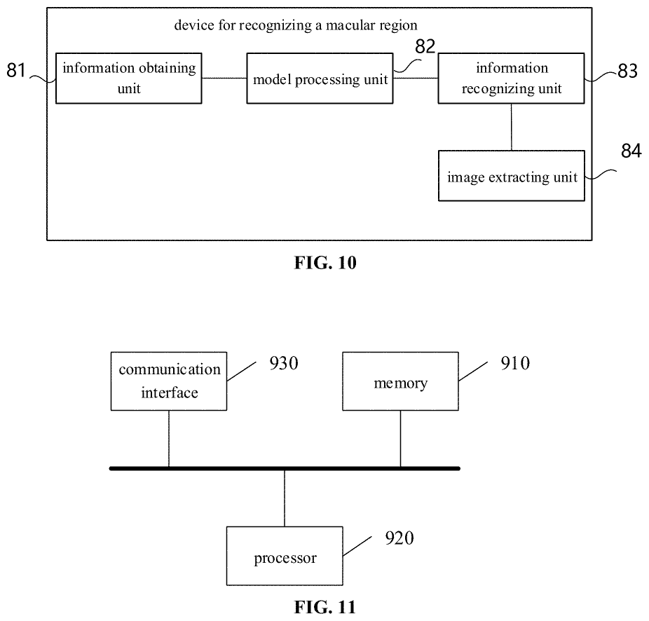

As shown in FIG. 10, a device for recognizing a macular region is provided according to another embodiment of the present application, the device may include:

an information obtaining unit 81 configured to obtain a fundus image of a target object; and extract blood vessel information and optic disc information from the fundus image;

a model processing unit 82 configured to input the blood vessel information and the optic disc information into a regression model to obtain location information of a macular fovea; and

an information recognizing unit 83 configured to determine location information of the macular region of an eye of the target object, based on the location information of the macula fovea.

In one possible implementation, the model processing unit 82 is configured to:

obtain at least one historical fundus image; obtain historical blood vessel information, historical optic disc information, and historical location information of the macular fovea based on the at least one historical fundus image; and train the regression model by taking the historical blood vessel information and the historical optic disc information as input parameters of the regression model and taking the historical location information of the macular fovea as an output parameter of the regression model.

In one possible implementation, the historical blood vessel information includes: historical coordinates of a blood vessel barycenter, and historical coordinates of a convergence point of at least two blood vessels; and

the historical optic disc information includes: a historical center of an optic disc, a historical horizontal diameter of the optic disc, and a historical vertical diameter of the optic disc.

In one possible implementation, the information obtaining unit 81 is configured to:

determine a center of the optic disc, a horizontal diameter of the optic disc and a vertical diameter of the optic disc based on the fundus image; obtain a location information set of a blood vessel from the fundus image, wherein the location information in the location information sets of different blood vessels is at least partially different; and determine coordinates of a blood vessel barycenter of the blood vessel based on the location information set of the blood vessel, and determining coordinates where an overlapping region of multiple blood vessels is the largest, as coordinates of a convergence point.

In one possible implementation, the information recognizing unit 83 is configured to:

determine a radius of the macular region based on the optic disc information; and

determine the location information of the macular region by taking the location information of the macula fovea as a center point based on the radius of the macular region.

In one possible implementation, the device further includes:

an image extracting unit 84, configured to generate a mask based on the location information of the macular region of the eye of the target object; and obtain an image at the macular region of the eye of the target object based on the mask and the fundus image of the target object.

It should also be understood that each unit in the above device may be provided in the terminal apparatus or in the network apparatus. When each unit of the device is provided in the network apparatus, the network apparatus may further include a communication unit, and at least one fundus image may be received through the communication unit, and the image of the macular region may be sent to the terminal apparatus through the communication unit.

In this embodiment, functions of modules in the device refer to the corresponding description of the above mentioned method and thus the description thereof is omitted herein.

In the embodiment, the blood vessel information and the optic disc information is extracted in the fundus image of the target object, the blood vessel information and the optic disc information are input into the regression model, and the location information of the macular region is determined based on the location information of the fovea of the macular region. In this way, the location of the macular fovea can be obtained by using the regress algorithm according to the blood vessel information and the optic disc information, which can effectively avoid the influence of illumination and lesion damage on the image quality of the macular region. The accuracy is improved, and the robustness of the extraction algorithm of the macular region is enhanced.

FIG. 11 shows a structural block diagram of a device for recognizing a macular region according to an embodiment of the present application. As shown in FIG. 11, the apparatus includes a memory 910 and a processor 920. The memory 910 stores a computer program executable on the processor 920. When the processor 920 executes the computer program, the method for recognizing a macular region in the foregoing embodiments is implemented. The number of the memory 910 and the processor 920 may be one or more.

The device/apparatus/terminal apparatus/server further includes:

a communication interface 930 configured to communicate with an external apparatus and exchange data.

The memory 910 may include a high-speed RAM memory and may also include a non-volatile memory, such as at least one magnetic disk memory.

If the memory 910, the processor 920, and the communication interface 930 are implemented independently, the memory 910, the processor 920, and the communication interface 930 may be connected to each other through a bus and communicate with one another. The bus may be an Industry Standard Architecture (ISA) bus, a Peripheral Component Interconnect (PCI) bus, an Extended Industry Standard Component (EISA) bus, or the like. The bus may be divided into an address bus, a data bus, a control bus, and the like. For ease of illustration, only one bold line is shown in FIG. 11, but it does not mean that there is only one bus or one type of bus.

Optionally, in a specific implementation, if the memory 910, the processor 920, and the communication interface 930 are integrated on one chip, the memory 910, the processor 920, and the communication interface 930 may implement mutual communication through an internal interface.

According to an embodiment of the present application, a computer-readable storage medium is provided for storing computer software instructions, which include programs involved in execution of the above the mining method.

In the description of the specification, the description of the terms “one embodiment,” “some embodiments,” “an example,” “a specific example,” or “some examples” and the like means the specific features, structures, materials, or characteristics described in connection with the embodiment or example are included in at least one embodiment or example of the present application. Furthermore, the specific features, structures, materials, or characteristics described may be combined in any suitable manner in any one or more of the embodiments or examples. In addition, different embodiments or examples described in this specification and features of different embodiments or examples may be incorporated and combined by those skilled in the art without mutual contradiction.

In addition, the terms “first” and “second” are used for descriptive purposes only and are not to be construed as indicating or implying relative importance or implicitly indicating the number of indicated technical features. Thus, features defining “first” and “second” may explicitly or implicitly include at least one of the features. In the description of the present application, “a plurality of” means two or more, unless expressly limited otherwise.

Any process or method descriptions described in flowcharts or otherwise herein may be understood as representing modules, segments or portions of code that include one or more executable instructions for implementing the steps of a particular logic function or process. The scope of the preferred embodiments of the present application includes additional implementations where the functions may not be performed in the order shown or discussed, including according to the functions involved, in substantially simultaneous or in reverse order, which should be understood by those skilled in the art to which the embodiment of the present application belongs.

Logic and/or steps, which are represented in the flowcharts or otherwise described herein, for example, may be thought of as a sequencing listing of executable instructions for implementing logic functions, which may be embodied in any computer-readable medium, for use by or in connection with an instruction execution system, device, or apparatus (such as a computer-based system, a processor-included system, or other system that fetch instructions from an instruction execution system, device, or apparatus and execute the instructions). For the purposes of this specification, a “computer-readable medium” may be any device that may contain, store, communicate, propagate, or transport the program for use by or in connection with the instruction execution system, device, or apparatus. More specific examples (not a non-exhaustive list) of the computer-readable media include the following: electrical connections (electronic devices) having one or more wires, a portable computer disk cartridge (magnetic device), random access memory (RAM), read only memory (ROM), erasable programmable read only memory (EPROM or flash memory), optical fiber devices, and portable read only memory (CDROM). In addition, the computer-readable medium may even be paper or other suitable medium upon which the program may be printed, as it may be read, for example, by optical scanning of the paper or other medium, followed by editing, interpretation or, where appropriate, process otherwise to electronically obtain the program, which is then stored in a computer memory.

It should be understood that various portions of the present application may be implemented by hardware, software, firmware, or a combination thereof. In the above embodiments, multiple steps or methods may be implemented in software or firmware stored in memory and executed by a suitable instruction execution system. For example, if implemented in hardware, as in another embodiment, they may be implemented using any one or a combination of the following techniques well known in the art: discrete logic circuits having a logic gate circuit for implementing logic functions on data signals, application specific integrated circuits with suitable combinational logic gate circuits, programmable gate arrays (PGA), field programmable gate arrays (FPGAs), and the like.

Those skilled in the art may understand that all or some of the steps carried in the methods in the foregoing embodiments may be implemented by a program instructing relevant hardware. The program may be stored in a computer-readable storage medium, and when executed, one of the steps of the method embodiment or a combination thereof is included.

In addition, each of the functional units in the embodiments of the present application may be integrated in one processing module, or each of the units may exist alone physically, or two or more units may be integrated in one module. The above-mentioned integrated module may be implemented in the form of hardware or in the form of software functional module. When the integrated module is implemented in the form of a software functional module and is sold or used as an independent product, the integrated module may also be stored in a computer-readable storage medium. The storage medium may be a read only memory, a magnetic disk, an optical disk, or the like.

The foregoing descriptions are merely specific embodiments of the present application, but not intended to limit the protection scope of the present application. Those skilled in the art may easily conceive of various changes or modifications within the technical scope disclosed herein, all these should be covered within the protection scope of the present application. Therefore, the protection scope of the present application should be subject to the protection scope of the claims.

What is claimed is:

1. A method for recognizing a macular region, comprising:
obtaining a fundus image of a target object;
extracting blood vessel information and optic disc information from the fundus image;
inputting the blood vessel information and the optic disc information into a regression model to obtain location information of a macular fovea; and
determining location information of the macular region of an eye of the target object, based on the location information of the macula fovea.

2. The method according to claim 1, further comprising:
obtaining at least one historical fundus image;
obtaining historical blood vessel information, historical optic disc information, and historical location information of the macular fovea based on the at least one historical fundus image; and
training the regression model by taking the historical blood vessel information and the historical optic disc information as input parameters of the regression model and taking the historical location information of the macular fovea as an output parameter of the regression model.

3. The method according to claim 2, wherein the historical blood vessel information comprises: historical coordinates of a blood vessel barycenter, and historical coordinates of a convergence point of at least two blood vessels, and
the historical optic disc information comprises: a historical center of an optic disc, a historical horizontal diameter of the optic disc, and a historical vertical diameter of the optic disc.

4. The method according to claim 1, wherein the extracting blood vessel information and optic disc information from the fundus image comprises:
determining a center of the optic disc, a horizontal diameter of the optic disc and a vertical diameter of the optic disc based on the fundus image;
obtaining a location information set of a blood vessel from the fundus image, wherein the location information in location information sets of different blood vessels is at least partially different; and
determining coordinates of a blood vessel barycenter of the blood vessel based on the location information set of the blood vessel, and determining coordinates where an overlapping region of multiple blood vessels is the largest, as coordinates of a convergence point.

5. The method according to claim 1, wherein the determining location information of the macular region of an eye of the target object, based on the location information of the macula fovea comprises:
determining a radius of the macular region based on the optic disc information; and
determining the location information of the macular region by taking the location information of the macula fovea as a center point based on the radius of the macular region.

6. The method according to claim 1, further comprising:
generating a mask based on the location information of the macular region of the eye of the target object; and
obtaining an image at the macular region of the eye of the target object based on the mask and the fundus image of the target object.

7. A device for recognizing a macular region, comprising one or more processors; and
a non-transitory storage device configured to store computer executable instructions, wherein
the computer executable instructions, when executed by the one or more processors, cause the one or more processors to:
obtain a fundus image of a target object;
extract blood vessel information and optic disc information from the fundus image;
input the blood vessel information and the optic disc information into a regression model to obtain location information of a macular fovea; and
determine location information of the macular region of an eye of the target object, based on the location information of the macula fovea.

8. The device according to claim 7, wherein the computer executable instructions, when executed by the one or more processors, cause the one or more processors further to:
obtain at least one historical fundus image;
obtain historical blood vessel information, historical optic disc information, and historical location information of the macular fovea based on the at least one historical fundus image; and
train the regression model by taking the historical blood vessel information and the historical optic disc information as input parameters of the regression model and taking the historical location information of the macular fovea as an output parameter of the regression model.

9. The device according to claim 8, wherein the historical blood vessel information comprises: historical coordinates of a blood vessel barycenter, and historical coordinates of a convergence point of at least two blood vessels, and
the historical optic disc information comprises: a historical center of an optic disc, a historical horizontal diameter of the optic disc, and a historical vertical diameter of the optic disc.

10. The device according to claim 7, wherein the computer executable instructions, when executed by the one or more processors, cause the one or more processors further to:
determine a center of the optic disc, a horizontal diameter of the optic disc and a vertical diameter of the optic disc based on the fundus image;
obtain a location information set of a blood vessel from the fundus image, wherein the location information in location information sets of different blood vessels is at least partially different; and
determine coordinates of a blood vessel barycenter of the blood vessel based on the location information set of the blood vessel, and determine coordinates where an overlapping region of multiple blood vessels is the largest, as coordinates of a convergence point.

11. The device according to claim 7, wherein the one or more programs, when executed by the one or more processors, cause the one or more processors further to:
determine a radius of the macular region based on the optic disc information; and
determine the location information of the macular region by taking the location information of the macula fovea as a center point based on the radius of the macular region.

12. The device according to claim 7, wherein the one or more programs, when executed by the one or more processors, cause the one or more processors further to:
generate a mask based on the location information of the macular region of the eye of the target object; and
obtain an image at the macular region of the eye of the target object based on the mask and the fundus image of the target object.

13. A non-transitory, computer-readable media having instructions encoded thereon, the instructions, when executed by a processor, are operable to:
obtain a fundus image of a target object;
extract blood vessel information and optic disc information from the fundus image;
input the blood vessel information and the optic disc information into a regression model to obtain location information of a macular fovea; and
determine location information of the macular region of an eye of the target object, based on the location information of the macula fovea.
